(19) United States
(12) Patent Application Publication
(10) Pub. No.: US 2023/0038151 A1
(43) Pub. Date: Feb. 9, 2023

(54) METHODS OF DETERMINING VIRAL TITER

(71) Applicant: LONZA WALKERSVILLE, INC., Walkersville, MD (US)

(72) Inventors: Young SHIN, Walkersville, MD (US); Anandita SETH, Walkersville, MD (US)

(21) Appl. No.: 17/758,834

(22) PCT Filed: Jan. 26, 2021
(86) PCT No.: PCT/US2021/014987

§ 371 Date: Jul. 14, 2022

Related U.S. Application Data
(60) Provisional application No. 62/966,639, filed on Jan. 28, 2020.

(51) Int. Cl. C12Q 1/70

(52) U.S. Cl. CPC C12Q 1/701; C12Q 1/6851

(57) ABSTRACT

The present disclosure relates to methods for determining a viral titer of a biological sample, suitably from a mammalian cell sample. The methods include the use of mechanical disruption of the cells, followed by droplet digital polymerase chain reaction (ddPCR) to determine the viral titer. Methods of mechanical disruption suitably include the use of glass beads.

### FIELD OF THE INVENTION

The present disclosure relates to methods for determining a viral titer of a biological sample, suitably from a mammalian cell sample. The methods include the use of mechanical disruption of the cells, followed by droplet digital polymerase chain reaction (ddPCR) to determine the viral titer. Methods of mechanical disruption suitably include the use of glass beads.

### BACKGROUND OF THE INVENTION

Lentivirus (LV) is one of the most popular delivery vehicles in cell and gene therapies. Similarly, adeno-associated virus (AAV) has also found uses as a gene therapy vehicle. The accurate measurement of infectious titer is an absolute requisite in the process of manufacturing, purification, and application of viral vectors. Conventional assay methods measuring viral titers, such as flow cytometry or quantitative polymerase chain reaction (qPCR), have some major drawbacks. For these assays, one needs to have a reporter or a specific antibody for the measurement of infectious titers. In addition, it is necessary to optimize the primers, probes, and standards in the qPCR assay before putting them into the assay, which is a very cumbersome process.

Droplet digital polymerase chain reaction (ddPCR) has emerged as a reliable, cutting-edge technology to quantify the absolute copy number of any gene of interest without using a standard curve. The RNA genome of LV is first reverse-transcribed to its cDNA before it integrates into the host chromosome. Therefore, infectious titers of LV can be determined using ddPCR by measuring the integration frequency of the transgene into the chromosomes of target cells. AAV viral vector can also be measureding using ddPCR. However, current methods to determine viral titers by ddPCR are rate-limited due to the tedious process of genomic DNA isolation, which involves extracting chromosomal DNA from a large number of virally-transduced cells.

What is needed therefore is a high-throughput method, which eliminates the genomic DNA extraction during sample preparations for ddPCR applications, and also eliminates the use of various potentially contaminating buffers and solutions. The present invention fulfills these needs.

### SUMMARY OF THE INVENTION

In some embodiments, provided herein is a method of determining a viral titer in a biological sample, comprising: obtaining the biological sample which contains a virally-transduced cell; mechanically disrupting the virally-transduced cell of the biological sample; conducting droplet digital polymerase chain reaction (ddPCR) on nucleic acid molecules removed from the disrupted virally-transduced cell; and calculating the viral titer.

In additional embodiments, provided herein is a method of determining a viral titer in a biological sample, consisting essentially of: obtaining the biological sample which contains a virally-transduced cell; mechanically disrupting the virally-transduced cell of the biological sample with glass beads; conducting droplet digital polymerase chain reaction (ddPCR) on nucleic acid molecules removed from the disrupted virally-transduced cell; and calculating the viral titer.

### BRIEF DESCRIPTION OF THE DRAWINGS

FIG. 1 shows lentiviral titer comparison between three methods, as described herein.

### DETAILED DESCRIPTION OF THE INVENTION

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the method/device being employed to determine the value. Typically the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation.

The use of the term “or” in the claims is used to mean “and/or” unless explicitly indicated to refer only to alternatives or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited, elements or method steps. It is contemplated that any embodiment discussed in this specification can be implemented with respect to any method, system, host cells, expression vectors, and/or composition of the invention. Furthermore, compositions, systems, cells, and/or nucleic acids of the invention can be used to achieve any of the methods as described herein.

As used herein, “nucleic acid,” “nucleic acid molecule,” or “oligonucleotide” means a polymeric compound comprising covalently linked nucleotides. The term “nucleic acid” includes polyribonucleic acid (RNA) and polydeoxyribonucleic acid (DNA), both of which may be single- or double-stranded. DNA includes, but is not limited to, complimentary DNA (cDNA), genomic DNA, plasmid or vector DNA, and synthetic DNA. RNA includes, but is not limited to, mRNA, tRNA, rRNA, snRNA, microRNA, miRNA, or MIRNA.

A “gene” as used herein refers to an assembly of nucleotides that encode a polypeptide, and includes cDNA and genomic DNA nucleic acid molecules. “Gene” also refers to a nucleic acid fragment that can act as a regulatory sequence preceding (5′ non-coding sequences) and following (3′ non-coding sequences) the coding sequence. In some embodiments, genes are integrated with multiple copies. In some embodiments, genes are integrated at predefined copy numbers.

### Methods of Determining Viral Titer

In exemplary embodiments, provided herein is a method of determining a viral titer in a biological sample. As used herein “viral titer” refers to a numeric expression of the quantity of a virus in a given volume, generally expressed as viral particles, transducing units, or infections particles, per milliliter (mL). Thus, the methods described herein that determine a viral titer are quantitative, in that they determine the actual number of viral particles, rather than simply qualitative measurements.

As used herein a “biological sample” refers to a solution or suspension, or a solution or suspension that has been dried prior to reconstitution, of cells or tissues that may contain a viral vector. Suitably, the biological sample is a cell solution that contains at least one virally-transduced cell.

As used herein, a “virally-transduced cell” is a cell into which a viral vector has been inserted, either transiently (inserted without integrating into the genome) or genomically integrated (inserted into the genome of the cell). As used herein, a “vector” or “expression vector” is a replicon, such as a plasmid, phage, virus, or cosmid, to which a nucleic acid molecule may be attached to bring about the replication and/or expression of the attached nucleic acid molecule in a cell. “Vector” includes episomal (e.g., plasmids) and non-episomal vectors. The term “vector” includes both viral and nonviral means for introducing a nucleic acid molecule into a cell in vitro, in vivo, or ex vivo. The term vector may include synthetic vectors. Vectors may be introduced into the desired cells by well-known methods, including, but not limited to, transfection, transduction, cell fusion, and lipofection. Vectors can comprise various regulatory elements including promoters.

“Transduction” as used herein means the introduction of an exogenous nucleic acid molecule, including a vector, into a cell, and includes transfection (e.g., use of lipid or polymer-based carriers, as well as mechanical transfection, electroporation) and viral transduction. A “transfected” cell comprises an exogenous nucleic acid molecule inside the cell and a “transformed” cell is one in which the exogenous nucleic acid molecule within the cell induces a phenotypic change in the cell. The transfected nucleic acid molecule can be integrated into the host cell's genomic DNA and/or can be maintained by the cell, temporarily or for a prolonged period of time, extra-chromosomally (transiently). Host cells or organisms that express exogenous nucleic acid molecules or fragments are referred to as “recombinant,” “transformed,” or “transgenic” organisms. A number of transfection techniques are generally known in the art. See, e.g., Graham et al., Virology, 52:456 (1973); Sambrook et al., Molecular Cloning, a laboratory manual, Cold Spring Harbor Laboratories, New York (1989); Davis et al., Basic Methods in Molecular Biology, Elsevier (1986); and Chu et al., Gene 13:197 (1981), the disclosures of each of which are incorporated by reference herein in their entireties. Suitably, transfection of a mammalian cell with one or more vectors utilizes a transfection agent, such as polyethyleneimine (PEI) or other suitable agent, including various lipids and polymers, to integrate the nucleic acids into the host cell's genomic DNA.

The methods for determining a viral titer include obtaining the biological sample which contains a virally-transduced cell. Biological samples can be obtained from laboratory settings, or large scale batch processes, or other suitable settings, and include samples that are prepared and then measured as described herein, as well as biological samples that are prepared in other settings or areas, stored, potentially shipped, and then measured using the methods described herein.

The methods further include mechanically disrupting the virally-transduced cell of the biological sample. As used herein, “mechanically disrupting” or “mechanical disruption” refers to the application of a force to the biological sample, that is not inherent to the sample, efficient to break or lyse the cells contained therein. Exemplary mechanical disruption techniques include the use of disruption with glass beads, sonication (including the use of a sonication bath as well as sonication tip/probe or ultrasound tip/probe), high power vortexing or mixing, application of shear forces via glass or plastic plates, the use of grinding, blending, mechanical homogenizers, etc.

In suitable embodiments, the mechanical disruption occurs via disruption with glass beads. In such methods, the biological sample, including the virally-transduced cells, is contacted with a solution of glass beads, vortexed for about 1 minute, and then vortexed again for 3-5 additional times, each for about 1 minute. Additional times and number of repetitions of vortexing can also be used. Glass beads for use in the methods described herein include silica beads from COLE-PARMER® (Vernon Hills, Ill.), and suitably have a diameter of about 100 μm-1 mm, more suitably about 100 μm, about 500 μm, or about 1 mm beads. Other material beads, such as zirconium beads, can also be utilized. Prior to use in the biological samples, the glass beads are suitably soaked in an acid solution (e.g., HCl), rinsed well with deionized water, and then baked above 150° C. for 12-24 hours to fully dry them. The beads are then chilled at 4° C. or on ice for about 30 minutes or more, prior to use, to fully cool. Acid washing and heat treatment can also be eliminated if beads are purchased pre-treated, and suitably are free from nuclease.

As described herein, the methods suitably exclude lysing the virally-transduced cells of the biological sample with a detergent or lysis buffer. As described herein, it has been determined that the use of such detergents and lysis buffers are not required, and by their elimination, costs, time of sample preparation and analysis can all be reduced, and also contamination from byproducts, unwanted debris or bacteria, as well as potential nucleases in the buffers, can also be reduced or eliminated.

Following the mechanical disruption of the virally-transduced cell, droplet digital polymerase chain reaction (ddPCR) is conducted on the nucleic acid molecules removed from the disrupted cells. As used herein, the nucleic acid molecules are “removed” from the disrupted cells simply by the action of the cells lysing or breaking. Suitably, no further action is required to isolate the nucleic acid molecules, including DNA, from the disrupted cells and the crude lysate (disruptate) is applied directly to a ddPCR assay. As described herein, a ddPCR performs digital PCR that is based on water-oil emulsion droplet technology. A sample is fractionated into 20,000 droplets, and PCR amplification of the template molecules (DNA) occurs in each individual droplet. ddPCR technology uses reagents and workflows similar to those used for most standard TaqMan probe-based assays. Exemplary ddPCR analysis kits and assays are readily available from, for example, BIORAD® (Hercules, Calif.). In embodiments, an additional step of counting the cells prior to ddPCR can be included. Methods for conducting ddPCR to determine viral titer can be found in, for example, Dobnik et al., “Accurate Quantification and Characterization of Adeno-Associated Viral Vectors,” Frontiers in Microbiology 10: Article 1570 (2019); and Abachin et al., “Comparison of reverse-transcriptase qPCR and droplet digital PCR for the quantification of dengue virus nucleic acid,” Biologicals 52:49-54 (2018), the disclosures of each of which are incorporated by reference herein in their entireties, particularly for the methods of ddPCR disclosed therein.

Based on the ddPCR analysis, the viral titer is then calculated. Calculation of the viral titer is readily carried out from the ddPCR analysis and results output. The infectious viral titer from ddPCR can be calculated by using a formula, TU/mL=F×C×D/V, where TU/mL is transducing units/mL, F is fraction of the cells transduced, C is the number of cells put in the assay at the time of transduction, D is the fold of dilution of virus inoculum, and V is the volume (mL) of virus inoculum put in the assay.

For example, assume 20% of cells were found to be transduced in the assay in which one thousand cells were seeded at the time of inoculation. The virus was diluted 100 fold before it was put in the assay and 0.1 mL was put in the assay. Then, the TU/mL=20×0.01×1,000×100/0.1=2.0E+05. To find the fraction of cells transduced (F), the total copy number of virus genome integrated into the chromosomes measured from ddPCR results is divided by the number of total cells at the time of harvest.

As described herein, suitably the virally-transduced cell that includes the viral vectors is a mammalian cell. As used herein, the term “mammalian cell” includes cells from any member of the order Mammalia, such as, for example, human cells, mouse cells, rat cells, monkey cells, hamster cells, and the like. In some embodiments, the cell is a mouse cell, a human cell, a Chinese hamster ovary (CHO) cell, a CHOK1 cell, a CHO-DXB11 cell, a CHO-DG44 cell, a CHOK1SV cell including all variants (e.g. POTELLIGENT®, Lonza, Slough, UK), a CHOK1SV GS-KO (glutamine synthetase knockout) cell including all variants (e.g., XCEED™ Lonza, Slough, UK). Exemplary human cells include human embryonic kidney (HEK) cells, such as HEK293, a HeLa cell, or a HT1080 cell.

Mammalian cells include mammalian cell cultures which can be either adherent cultures or suspension cultures. Adherent cultures refer to cells that are grown on a substrate surface, for example a plastic plate, dish or other suitable cell culture growth platform, and may be anchorage dependent. Suspension cultures refer to cells that can be maintained in, for example, culture flasks or large suspension vats, which allows for a large surface area for gas and nutrient exchange. Suspension cell cultures often utilize a stirring or agitation mechanism to provide appropriate mixing. Media and conditions for maintaining cells in suspension are generally known in the art. An exemplary suspension cell culture includes human HEK293 clonal cells.

As described herein, exemplary viral vector titers that can be determined using the methods provided include lentivirus viral titer and adeno-associated virus (AAV) viral titer, as well as other viral vector titers.

Lentiviral vector (LV) is a well studied vector system based on human immunodeficiency virus (HIV-1). Other lentiviral systems have also been developed as gene transfer systems, including HIV-2 simian immunodeficiency virus, nonprimate lentiviruses, feline immunodeficiency virus, and bovine immunodeficiency virus, etc. Guided by safety concerns due to the pathogenic nature of HIV-1 in humans, the most widely used lentiviral system for use in clinical and research and development purposes is based on the four-plasmid system that expresses:
1) Lentiviral group specific antigen (GAG) gene and a lentiviral polymerase (POL) protein
2) Envelope protein (usually Vesicular Somatitis Virus Glycoprotein (VSV-G))
3) HIV regulator of expression of virion proteins (Rev) protein; and
4) A Transfer vector (TV) containing a gene of interest (GOI)

Lentiviral vectors are generally produced with a gene of interest that is to be introduced into a desired cell for therapy and disease treatment, including immunodeficiencies and neurodegenerative diseases.

As used herein, the term “adeno-associated virus (AAV)” refers to a small sized, replicative-defective nonenveloped virus containing a single stranded DNA of the family Parvoviridae and the genus Dependoparvovirus. Over 10 adeno-associated virus serotypes have been identified so far, with serotype AAV2 being the best characterized. Other non-limiting examples of AAV serotypes are ANC80, AAV1, AAV3, AAV4, AAV5, AAV6, AAV7, AAV8, AAV9, AAV10, and AAV11. In addition to these serotypes, AAV pseudotypes have been developed. An AAV pseudotype contains the capsid of a first serotype and the genome of a second serotype (e.g. the pseudotype AAV2/5 would correspond to an AAV with the genome of serotype AAV2 and the capsid of AAV5).

As referred to herein, the term “adenovirus” refers to a nonenveloped virus with an icosahedral nucleocapsid containing a double stranded DNA of the family Adenoviridae. Over 50 adenoviral subtypes have been isolated from humans and many additional subtypes have been isolated from other mammals and birds. Birds. See, e.g., Ishibashi et al., “Adenoviruses of animals,” In The Adenoviruses, Ginsberg, ed., Plenum Press, New York, N.Y., pp. 497-562 (1984); Strauss, “Adenovirus infections in humans,” In The Adenoviruses, Ginsberg, ed., Plenum Press, New York, N.Y., pp. 451-596 (1984). These subtypes belong to the family Adenoviridae, which is currently divided into two genera, namely Mastadenovirus and Aviadenovirus. All adenoviruses are morphologically and structurally similar. In humans, however, adenoviruses show diverging immunological properties and are, therefore, divided into serotypes. Two human serotypes of adenovirus, namely AV2 and AV5, have been studied intensively and have provided the majority of general information about adenoviruses.

In further embodiments, provided herein is a method of determining a viral titer in a biological sample, consisting essentially of: obtaining the biological sample which contains a virally-transduced cell; mechanically disrupting the virally-transduced cell of the biological sample with glass beads; conducting droplet digital polymerase chain reaction (ddPCR) on nucleic acid molecules removed from the disrupted virally-transduced cell; and calculating the viral titer.

Methods described herein that “consist essentially of” the recited steps exclude steps the use a lysis buffer, detergent, or a detergent step or lysis step, and such steps are considered a material alteration to the methods that consist essentially of the recited steps and thus are specifically excluded from such methods. Suitably, a column purification step is also excluded from the methods that consist essentially of the recited steps.

Methods of producing virally-transduced cells that can be measuring using the methods described herein can be produced in any suitable reactor(s) including but not limited to stirred tank, airlift, fiber, microfiber, hollow fiber, ceramic matrix, fluidized bed, fixed bed, and/or spouted bed bioreactors. As used herein, “reactor” can include a fermenter or fermentation unit, or any other reaction vessel and the term “reactor” is used interchangeably with “fermenter.” The term fermenter or fermentation refers to both microbial and mammalian cultures. For example, in some aspects, an example bioreactor unit can perform one or more, or all, of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and CO₂ levels, maintenance of pH level, agitation (e.g., stirring), and/or cleaning/sterilizing. Example reactor units, such as a fermentation unit, may contain multiple reactors within the unit, for example the unit can have 1, 2, 3, 4, 5, 10, 15, 20, 25, 30, 35, 40, 45, 50, 60, 70, 80, 90, or 100, or more bioreactors in each unit and/or a facility may contain multiple units having a single or multiple reactors within the facility. In various embodiments, the bioreactor can be suitable for batch, semi fed-batch, fed-batch, perfusion, and/or a continuous fermentation processes. Any suitable reactor diameter can be used. In embodiments, the bioreactor can have a volume between about 100 mL and about 50,000 L. Non-limiting examples include a volume of 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, 100 liters, 150 liters, 200 liters, 250 liters, 300 liters, 350 liters, 400 liters, 450 liters, 500 liters, 550 liters, 600 liters, 650 liters, 700 liters, 750 liters, 800 liters, 850 liters, 900 liters, 950 liters, 1000 liters, 1500 liters, 2000 liters, 2500 liters, 3000 liters, 3500 liters, 4000 liters, 4500 liters, 5000 liters, 6000 liters, 7000 liters, 8000 liters, 9000 liters, 10,000 liters, 15,000 liters, 20,000 liters, and/or 50,000 liters. Additionally, suitable reactors can be multi-use, single-use, disposable, or non-disposable and can be formed of any suitable material including metal alloys such as stainless steel (e.g., 316L or any other suitable stainless steel) and Inconel, plastics, and/or glass.

### Additional Exemplary Embodiments

Embodiment 1 is a method of determining a viral titer in a biological sample, comprising: obtaining the biological sample which contains a virally-transduced cell; mechanically disrupting the virally-transduced cell of the biological sample; conducting droplet digital polymerase chain reaction (ddPCR) on nucleic acid molecules removed from the disrupted virally-transduced cell; and calculating the viral titer.

Embodiment 2 includes the method of embodiment 1, wherein the method does not include lysing the virally-transduced cell with a detergent or lysis buffer.

Embodiment 3 includes the method of embodiments 1 or 2, wherein the mechanically disrupting comprises disruption with glass beads.

Embodiment 4 includes the method of embodiments 1 or 2, wherein the mechanically disrupting comprises sonication.

Embodiment 5 includes the method of any one of embodiments 1-4, wherein the virally-transduced cell is a mammalian cell.

Embodiment 6 includes the method of embodiment 5, wherein the mammalian cell is a human cell.

Embodiment 7 includes the method of embodiment 6, wherein the human cell is a human embryonic kidney (HEK) cell.

Embodiment 8 includes the method of embodiment 7, wherein the viral titer is an adeno-associated virus (AAV) viral titer.

Embodiment 9 includes the method of embodiment 7, wherein the viral titer is a lentivirus viral titer.

Embodiment 10 includes the method of embodiment 5, wherein the mammalian cell is a Chinese hamster ovary (CHO) cell.

Embodiment 11 includes the method of embodiment 10, wherein the viral titer is an adeno-associated virus (AAV) viral titer.

Embodiment 12 includes the method of embodiment 10, wherein the viral titer is a lentivirus viral titer.

Embodiment 13 is a method of determining a viral titer in a biological sample, consisting essentially of: obtaining the biological sample which contains a virally-transduced cell; mechanically disrupting the virally-transduced cell of the biological sample with glass beads; conducting droplet digital polymerase chain reaction (ddPCR) on nucleic acid molecules removed from the disrupted virally-transduced cell; and calculating the viral titer.

Embodiment 14 includes the method of embodiment 13, wherein the virally-transduced cell is a mammalian cell.

Embodiment 15 includes the method of embodiment 14, wherein the mammalian cell is a human cell.

Embodiment 16 includes the method of embodiment 15, wherein the human cell is a human embryonic kidney (HEK) cell.

Embodiment 17 includes the method of embodiment 16, wherein the viral titer is an adeno-associated virus (AAV) viral titer.

Embodiment 18 includes the method of embodiment 16, wherein the viral titer is a lentivirus viral titer.

Embodiment 19 includes the method of embodiment 14, wherein the mammalian cell is a Chinese hamster ovary (CHO) cell.

Embodiment 20 includes the method of embodiment 19, wherein the viral titer is an adeno-associated virus (AAV) viral titer.

Embodiment 21 includes the method of embodiment 19, wherein the viral titer is a lentivirus viral titer.

### EXAMPLES

### Example 1

### High Throughput Format for Measurement of Viral Titer

To avoid the tedious DNA extraction process, which commonly involves detergent-mediated cell lysis and column purification of the DNA thereafter, the cells are instead mechanically disrupted using glass beads.

The crude lysates prepared from the cells transduced with lentivirus (LV) encoding green fluorescent protein, GFP were applied directly to a ddPCR assay. To compare and validate this approach with conventional methods, DNA was also isolated from LV transduced cells by using a commercially available kit from Qiagen (QlAamp DNA Blood Mini Kit). The primer-probe sets specific to long terminal repeat (LTR) region of LV and beta-actin sequence of the host were used to amplify the target sequences. To calculate the infectious titers, the following three methods were compared:
1. Sample DNA for ddPCR was isolated using the Qiagen kit. The cell number in the corresponding sample was calculated from the copy number of beta-actin in the same sample, based on which LV titer was calibrated.
2. Sample DNA for ddPCR was isolated using the Qiagen kit. RNase A was included during the isolation procedure to remove any cellular RNA. The cell number in the corresponding sample was calculated from the DNA amount in the same sample, based on which LV titer was calibrated.
3. Crude cell lysates were prepared by disrupting the cells using glass beads and directly applied to ddPCR. The cell number in the corresponding sample was directly counted before the disruption of cells by using ViCell, based on which LV titer was calibrated.

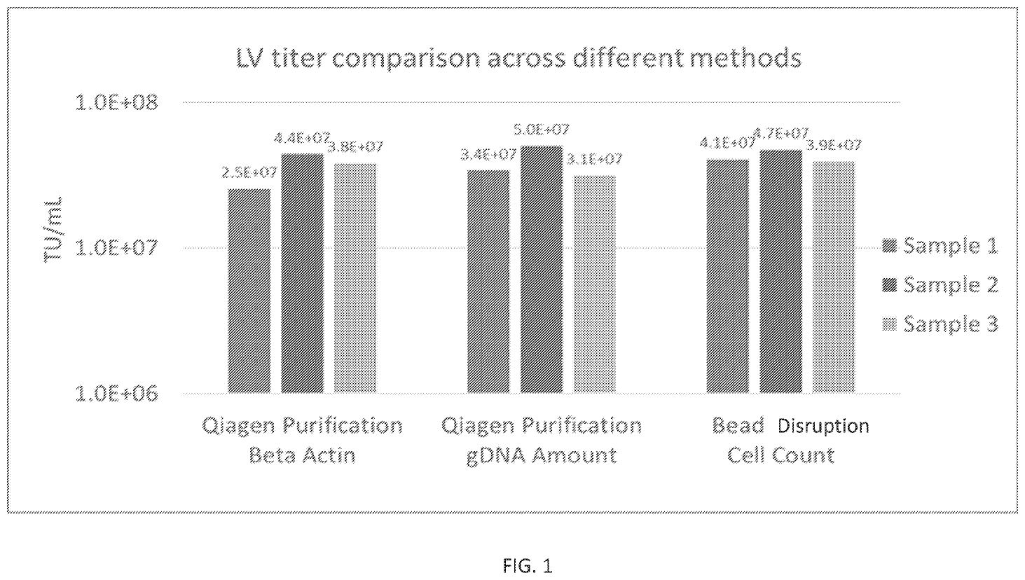

Cells in the 6-well culture plate were transduced with LV-GFP and processed by the three different methods above. Three samples for ddPCR were prepared for each method. The LV titers from these samples are calculated and presented in FIG. 1 as transducing units (TU)/mL. Table 1 below summarizes the results with statistical analysis.

TABLE 1
Summary of Viral Titer Calculation

| | Qiagen | Qiagen | Bead |
|---|---|---|---|
| | purification- | purification- | disruption- |
| | Beta actin | gDNA amount | Cell count |
| Sample 1 | 2.5E7 | 3.4E7 | 4.1E7 |
| Sample 2 | 4.4E7 | 5.0E7 | 4.7E7 |
| Sample 3 | 3.8E7 | 3.1E7 | 3.9E7 |
| Average (TU/mL) | 3.6E7 | 3.8E7 | 4.2E7 |
| Standard Deviation (STD) | 7.7E6 | 8.3E6 | 3.5E6 |
| Coefficient of variation (CV) | 21.6% | 21.6% | 8.2% |

The infectious LV titers calculated from the three different methods are comparable to each other for the 3 samples tested, indicating that crude cell lysate prepared by bead disruption is sufficient for direct ddPCR application. Also, the coefficient of variation (CV) from the third method (bead disruption—cell count) is significantly smaller (8.2%) than the others, suggesting that it is more consistent and reproducible.

It will be readily apparent to one of ordinary skill in the relevant arts that other suitable modifications and adaptations to the methods and applications described herein can be made without departing from the scope of any of the embodiments.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

All publications, patents and patent applications mentioned in this specification are herein incorporated by reference to the same extent as if each individual publication, patent or patent application was specifically and individually indicated to be incorporated by reference.

1. A method of determining a viral titer in a biological sample, comprising:
a. obtaining the biological sample which contains a virally-transduced cell;
b. mechanically disrupting the virally-transduced cell of the biological sample;
c. conducting droplet digital polymerase chain reaction (ddPCR) on nucleic acid molecules removed from the disrupted virally-transduced cell; and
d. calculating the viral titer.

2. The method of claim 1, wherein the method does not include lysing the virally-transduced cell with a detergent or lysis buffer.

3. The method of claim 1, wherein the mechanically disrupting comprises disruption with glass beads.

4. The method of claim 1, wherein the mechanically disrupting comprises sonication.

5. The method of claim 1, wherein the virally-transduced cell is a mammalian cell.

6. The method of claim 5, wherein the mammalian cell is a human cell.

7. The method of claim 6, wherein the human cell is a human embryonic kidney (HEK) cell.

8. The method of claim 7, wherein the viral titer is an adeno-associated virus (AAV) viral titer.

9. The method of claim 7, wherein the viral titer is a lentivirus viral titer.

10. The method of claim 5, wherein the mammalian cell is a Chinese hamster ovary (CHO) cell.

11. The method of claim 10, wherein the viral titer is an adeno-associated virus (AAV) viral titer.

12. The method claim 10, wherein the viral titer is a lentivirus viral titer.

13. A method of determining a viral titer in a biological sample, consisting essentially of:
a. obtaining the biological sample which contains a virally-transduced cell;
b. mechanically disrupting the virally-transduced cell of the biological sample with glass beads;
c. conducting droplet digital polymerase chain reaction (ddPCR) on nucleic acid molecules removed from the disrupted virally-transduced cell; and
d. calculating the viral titer.

14. The method of claim 13, wherein the virally-transduced cell is a mammalian cell.

15. The method of claim 14, wherein the mammalian cell is a human cell.

16. The method of claim 15, wherein the human cell is a human embryonic kidney (HEK) cell.

17. The method of claim 16, wherein the viral titer is an adeno-associated virus (AAV) viral titer.

18. The method claim 16, wherein the viral titer is a lentivirus viral titer.

19. The method of claim 14, wherein the mammalian cell is a Chinese hamster ovary (CHO) cell.

20. The method of claim 19, wherein the viral titer is an adeno-associated virus (AAV) viral titer or wherein the viral titer is a lentivirus viral titer.

21. (canceled)
